(19) United States
(12) Patent Application Publication
(10) Pub. No.: US 2026/0306690 A1
(43) Pub. Date: Oct. 8, 2026

(54) VOLATILE COMPOSITION DISPENSER AND METHODS OF USING SAME

(71) Applicant: The Procter & Gamble Company, Cincinnati, OH (US)

(72) Inventors: Matthew Stephen BAUER, Loveland, OH (US); Hua HU, Mason, OH (US); Ronald David TURNER, Walton, KY (US)

(21) Appl. No.: 19/640,735

(22) Filed: Apr. 7, 2026

Related U.S. Application Data
(60) Provisional application No. 63/928,046, filed on Dec. 1, 2025.
(60) Provisional application No. 63/785,018, filed on Apr. 8, 2025.

(51) Int. Cl. A61L 9/03; G01S 13/02; G01S 13/89

(52) U.S. Cl. CPC A61L 9/03; G01S 13/0209; G01S 13/89; A61L 2209/111; A61L 2209/12; A61L 2209/21

(57) ABSTRACT

Volatile composition dispensing systems and methods for coordinating and optimizing fragrance compatibility and reach across multiple fragrance-dispensing devices. The volatile composition dispensing systems and methods include evaluating and optimizing fragrance performance across two or more fragrance-dispensing devices based on spatial relationships between fragrance-dispensing devices and compositional aspects of respective volatile compositions.

### TECHNOLOGICAL FIELD

The present disclosure generally relates to volatile composition dispensing systems and methods for coordinating scent emission across multiple devices, and more particularly to volatile composition dispensing systems and methods for determining relationships (e.g., spatial relationships) between volatile composition dispensers and optimizing fragrance compatibility and dispensing parameters based on said relationships.

### BACKGROUND

Volatile composition dispensing systems, commonly known as scent diffusers or fragrance dispensers, have become increasingly popular for enhancing indoor environments in residential, commercial, and hospitality settings. These devices typically operate by heating or otherwise volatilizing fragrant compounds to release aromatic molecules into the surrounding air. Traditional scent dispensers operate independently without any coordination with other devices that may be present in the same space or adjacent areas. When multiple fragrance-emitting devices operate simultaneously in proximity to one another, several challenges can arise. Different fragrance compositions may interact in undesirable ways, creating conflicting or overwhelming scent profiles. Additionally, the effective range and intensity of fragrance projection can vary based on the chemical properties of different compounds, such as volatility, molecular weight, and odor detection thresholds. Further, environmental objects, such as walls, can affect how fragrances are dispensed and interact with a surrounding environment.

Current scent dispensing systems generally lack the ability to detect their spatial relationship to other devices or to automatically adjust operational parameters based on such spatial considerations. This limitation can result in suboptimal fragrance experiences, including scent conflicts, inefficient fragrance utilization, or inadequate coverage of the intended space. Additionally, existing systems typically do not account for the chemical compatibility of different fragrance compositions when multiple devices are operating in the same environment.

Accordingly, there exists a need for improved fragrance harmony in multi-device scent dispensing systems.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The present disclosure is directed to volatile composition dispensing systems and methods for coordinating the release of volatile compositions to optimize fragrance harmony. The volatile composition dispensing systems and methods may optimize fragrance harmony based on at least one of: (1) a spatial relationship between volatile composition dispensers, (2) a spatial relationship between volatile composition dispensers and a surrounding environment, and (3) compositional aspects of volatile compositions.

According to an example of the present disclosure, a volatile composition dispenser includes a transceiver and a controller. The controller is configured for determining a spatial relationship of the volatile composition dispenser relative to at least one of a surrounding environment and a second volatile composition dispenser and modifying at least one dispensing parameter based on the spatial relationship. The volatile composition dispenser is configured to dispense at least one volatile composition based on the at least one dispensing parameter.

According to another example of the present disclosure, a volatile composition dispensing system includes a first volatile composition dispenser and a second volatile composition dispenser. The first volatile composition dispenser includes a first volatile composition, a first ultra-wideband transceiver, and a first controller. The second volatile composition dispenser includes a second volatile composition, a second ultra-wideband transceiver, and a second controller. The first controller is configured for modifying at least one dispensing parameter of the first volatile composition dispenser based on at least one of: (1) a spatial relationship between the first composition dispenser relative to the second volatile composition dispenser, (2) compositional aspects of the second volatile composition, (3) a compatibility score of the first volatile composition and the second volatile composition, and (4) a unique identifier corresponding to the first volatile composition that is wirelessly communicated to the first controller.

In another example, a method of volatile composition dispensing is provided. The method includes capturing, via an ultra-wideband transceiver of a first volatile composition dispenser, ultra-wideband radar data, determining, via a controller operably connected to the ultra-wideband transceiver, a spatial relationship between the first volatile composition dispenser and a second volatile composition dispenser, wherein the spatial relationship is determined based on the ultra-wideband radar data, determining compositional aspects of a first volatile composition of the first volatile composition dispenser, determining compositional aspects of a second volatile composition of the second volatile composition dispenser, and adjusting, via the controller, at least one dispensing parameter of at least one of the first volatile composition dispenser and the second volatile composition dispenser based on at least one of the spatial relationship, the compositional aspects of the first volatile composition, and the compositional aspects of the second volatile composition.

### BRIEF DESCRIPTION OF THE DRAWINGS

The following detailed description of specific examples of the present disclosure can be best understood when read in conjunction with the following drawings, where like structure is indicated with like reference numerals and wherein:

FIG. 1 is a system diagram of a volatile composition dispensing system, in accordance with examples of the present disclosure.

FIG. 2 is a system diagram of a volatile composition dispensing system, in accordance with examples of the present disclosure.

FIG. 3 is a system diagram of a volatile composition dispensing system, in accordance with examples of the present disclosure.

FIG. 4A illustrates a user interface of a volatile composition dispensing system, in accordance with examples of the present disclosure.

FIG. 4B illustrates a user interface of a volatile composition dispensing system, in accordance with examples of the present disclosure.

FIG. 4C illustrates a user interface of a volatile composition dispensing system, in accordance with examples of the present disclosure.

FIG. 5 illustrates a compatibility diagram of a volatile composition dispensing system, in accordance with examples of the present disclosure.

FIG. 6 illustrates a reach factor diagram of a volatile composition dispensing system, in accordance with examples of the present disclosure.

FIG. 7 illustrates an activation temperature diagram of a volatile composition dispensing system, in accordance with examples of the present disclosure.

FIG. 8 illustrates a perfume note compatibility matrix of a volatile composition dispensing system, in accordance with examples of the present disclosure.

### DETAILED DESCRIPTION

Reference within the specification to “form(s)”, “aspect(s)”, “embodiment(s)” or the like means that a particular material, feature, structure and/or characteristic described in connection with the form/aspect/embodiment is included in at least one form/aspect/embodiment, optionally several forms/aspects/embodiments, but it does not mean that all forms/aspects/embodiments incorporate the material, feature, structure, and/or characteristic described. Furthermore, materials, features, structures and/or characteristics may be combined in any suitable manner across different forms/aspects/embodiments, and materials, features, structures and/or characteristics may be omitted or substituted from what is described. Thus, forms/aspects/embodiments described herein may comprise or be combinable with elements or components of other forms/aspects/embodiments despite not being expressly exemplified in combination, unless otherwise stated or an incompatibility is stated.

### DEFINITIONS

The term "volatile compositions" as used herein, refers to a material that comprises a vaporizable material. The term "volatile compositions," thus includes (but is not limited to) compositions that are comprised entirely of a single volatile material. The terms "volatile materials," "aroma," "fragrance," and "scents," as used herein, include, but are not limited to pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile compositions including, but not limited to perfumes, aromatic materials, and scented materials, will often comprise one or more volatile materials (which may form a unique and/or discrete unit comprised of a collection of volatile materials). It should be understood that the term "volatile composition" refers to compositions that have at least one volatile component, and it is not necessary for all of the component materials of the volatile composition to be volatile. The volatile compositions described herein may, thus, also have non-volatile components. It should also be understood that when the volatile compositions are described herein as being "emitted," this refers to the volatilization of the volatile components thereof and does not require that the non-volatile components thereof be emitted. The volatile compositions of interest herein can be in any suitable form including, but not limited to, solids, liquids, gels, encapsulates, and combinations thereof.

As used herein, the term “ultra-wideband” or “UWB” refers, in a non-limiting sense, to wireless communication and/or sensing technology that employs radio signals having a large occupied bandwidth relative to the center frequency to enable high-resolution detection, ranging, and characterization of objects and environments. In various aspects, UWB signals may have a fractional bandwidth greater than about twenty percent (20%) of the center frequency and/or an absolute bandwidth greater than 500 MHz may be generated using short-duration pulses, multicarrier or spread-spectrum techniques, or other modulation schemes. For example, and without limitation, UWB signals may have a frequency range between about 3.1 GHz and about 10.6 GHz and/or a low-power spectral density (e.g., about -41.3 dBm/MHz). In another example, UWB sensing may provide centimeter-level ranging using Time-of-Flight (ToF) and Angle-of-Arrival (AoA) techniques. UWB sensing may be used for ranging, localization, radar, object detection, presence and motion sensing, imaging, material or biometric characterization, environmental mapping, and/or occupancy or gesture detection. References to UWB or ultra-wideband technology are intended to encompass any present or future implementation that provides wideband, low-power, short-range wireless signaling or sensing consistent with the foregoing description, without being limited to any particular regulatory definition, standard, protocol, modulation format, frequency band or application.

### VOLATILE COMPOSITION DISPENSING SYSTEM

The present disclosure is directed to volatile composition dispensing systems and methods for coordinating the release of volatile compositions to optimize fragrance harmony. The volatile composition dispensing systems and methods may optimize fragrance harmony based on one or more of: (1) a spatial relationship between volatile composition dispensers, (2) a spatial relationship between volatile composition dispensers and a surrounding environment, and (3) compositional aspects of volatile compositions.

A volatile composition dispensing system may include multiple volatile composition dispensers configured to create coordinated olfactory experiences across different zones or areas within a target environment. Each volatile composition dispenser may include components for storing, heating, and dispensing volatile compositions, along with communication capabilities for coordinating with other volatile composition dispensers in the volatile composition dispenser system.

The volatile composition dispensing system may use spatial relationship determination to optimize fragrance distribution based on the positioning of volatile composition dispensers relative to each other and to environmental objects. The volatile composition dispensing system may include ultra-wideband (UWB) transceivers that provide distance and angle measurements between dispensers and environmental features. This spatial mapping capability enables the volatile composition dispensing system to identify overlapping fragrance zones, physical barriers, and optimal positioning for creating a desired olfactory experience.

Fragrance compatibility assessment may be performed by evaluating interactions between different volatile compositions when dispensed simultaneously or sequentially. The volatile composition dispensing system may generate compatibility scores that quantify how well different fragrance profiles blend when released in a shared environment. These compatibility scores may be based on fragrance chemistry principles, sensory harmony data, and volatility characteristics of individual fragrance components.

The volatile composition dispensing system may adjust dispensing parameters automatically based on spatial relationships, compatibility assessments, and user preferences. Dispensing parameters may include temperature settings for selective volatilization, activation timing for coordinated release, intensity levels for balanced scent distribution, duration settings for temporal control, and selection of specific fragrance notes from multi-component volatile compositions.

A controller may manage coordination across multiple volatile composition dispensers by processing spatial mapping data, evaluating fragrance compatibility, and generating dispensing parameter commands. The controller may enable real-time adaptation to changing conditions within the environment, continuously monitoring spatial relationships and adjusting dispensing parameters to maintain optimal olfactory experiences as environmental conditions, device configurations, or positioning change over time.

The volatile composition dispensing system may include identification capabilities for determining specific volatile compositions housed in each dispenser. Cartridge identification may be accomplished through various technologies such as Near-Field Communication (NFC), radio frequency identification (RFID) tags, quick response (QR) codes, or other machine-readable identifiers that correspond to specific fragrance formulations and their associated chemical properties.

### VOLATILE COMPOSITION DISPENSING SYSTEM EXAMPLE

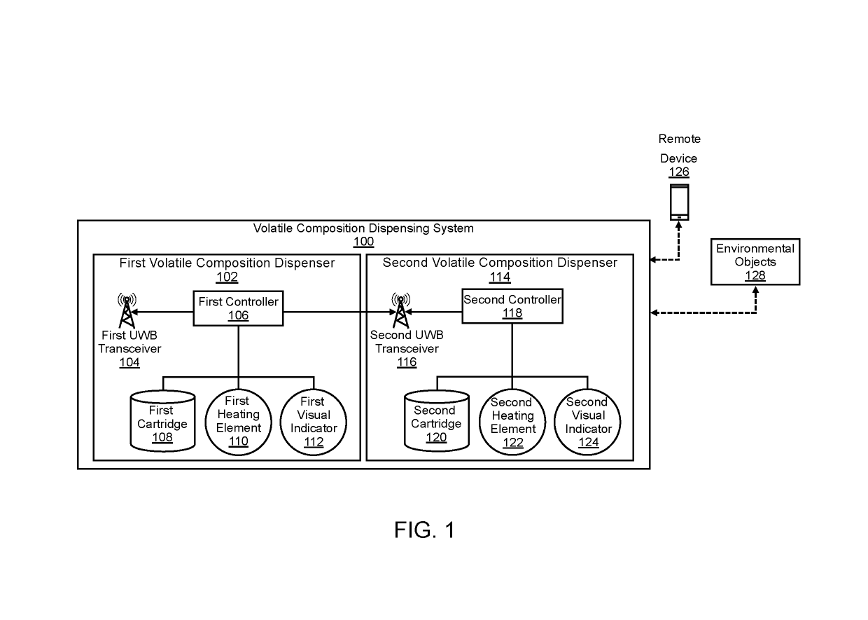

Referring to FIG. 1, a volatile composition dispensing system 100 is illustrated that demonstrates the coordinated operation of multiple volatile composition dispensers. The volatile composition dispensing system 100 comprises a first volatile composition dispenser 102 and a second volatile composition dispenser 114. The volatile composition dispensing system 100 may be configured to coordinate the operational and dispensing parameters of each volatile composition dispenser to create a harmonized olfactory environment based on a spatial relationship and fragrance compatibility. Example volatile composition dispensers are disclosed in U.S. Provisional Patent Application No. 63/892,897 and U.S. Patent Application No. 19/172,771, each of which is hereby incorporated herein by reference in its entirety.

The first volatile composition dispenser 102 may include an ultra-wideband (UWB) transceiver 104 that may provide spatial measurement and communication capabilities. This enables the first volatile composition dispenser 102 to determine its position relative to other volatile composition dispensers and environmental objects. A first controller 106 can function as a central processing unit for managing the operations of the first volatile composition dispenser 102. The first controller 106 can be configured for spatial analysis, fragrance compatibility analysis, and dispensing parameter optimization. The first controller 106 may be operably connected to each component within the first volatile composition dispenser 102.

A first volatile cartridge 108 may contain volatile compositions for dispensing and include identification mechanisms (e.g., near-field communication (NFC) tags) that enable the first controller 106 to determine specific fragrance notes and chemical characteristics of a volatile composition loaded into the first volatile composition dispenser 102. Each volatile composition dispenser may include one or more cartridges, and the cartridges may each include one or more different volatile compositions. Example cartridges are disclosed in U.S. Provisional Patent Application No. 63/892,897 and U.S. Patent Application No. 19/172,771. A first heating element 110 may provide temperature control capabilities for selective volatilization of fragrance compounds based on corresponding boiling points and volatility characteristics. A first visual indicator 112 may provide visual feedback regarding volatile composition dispenser status, fragrance compatibility, and operational parameters.

The volatile composition dispensing system 100 may include a second volatile composition dispenser 114. The volatile composition dispensers in the volatile composition dispensing system 100 may have the same or different forms (e.g., plug-in, corded, portable, etc.) and the same or different dispensing mechanisms (e.g., heater, fan, etc.). The second volatile composition dispenser 114 includes a second UWB transceiver 116, a second controller 118, a second volatile cartridge 120, a second heating element 122, and a second visual indicator 124. The second UWB transceiver 116 may enable bidirectional communication with the first UWB transceiver 104 and can be configured for determining a spatial relationship between the first volatile composition dispenser 102 and the second volatile composition dispenser 114. The second controller 118 can manage the operational and dispensing parameters of the second volatile composition dispenser 114 while coordinating with the first controller 106 to optimize fragrance dispensing for the entire volatile composition dispensing system 100.

A second cartridge 120 may house volatile compositions different from volatile compositions in the first cartridge 108, enabling the volatile composition dispensing system to create unique scent profiles through coordination dispensing of complementary or contrasting fragrance notes. In another example, the first cartridge 108 and second cartridge 120 may house the same volatile composition or same set of volatile compositions. A second heating element 122 can provide independent temperature control. The second heating element 122 may be coordinated with the first heating element 110 to achieve desired volatilization timing and intensity across the two volatile composition dispensers. A second visual indicator 124 provides coordinating lighting effects to create unified visual feedback regarding the volatile composition dispensing system operation.

The volatile composition dispensing system 100 may include a remote device 126 that may enable user interaction and system configuration through wireless communication with the first controller 106 and the second controller 118. The remote device 126 may allow adjustment of operational parameters, selection of fragrance combinations, and monitoring of system status without direct physical interaction with the first volatile composition dispenser 102 and the second volatile composition dispenser 114. As discussed above, the volatile composition dispensing system may include identification capabilities for determining specific volatile compositions housed in each dispenser. The remote device 126 may allow the user to manually select the specific volatile composition or cartridge that is present in a volatile composition dispenser.

Environmental objects 128 may represent physical barriers, furniture, walls, and other obstacles detected by the UWB transceivers during spatial analysis. The first UWB transceiver 104 and second UWB transceiver 116 may conduct continuous spatial sensing to monitor changes to the surrounding environment (e.g., new obstacle detection).

The first UWB transceiver 104 and second UWB transceiver 116 may be configured for two-way ranging operations and continuous spatial relationship monitoring. This enables real-time data communication regarding spatial measurements, composition identifiers, operational parameters, and synchronization signals that can be used for optimizing release of fragrance notes for a desired olfactory experience.

The volatile composition dispensing system can be configured to adapt to changes in spatial configuration, allowing volatile composition dispensers to automatically adjust operational parameters and fragrance dispensing parameters when volatile composition dispensers are moved, added, or removed from a surrounding environment or a network.

The volatile composition dispensing system may be configured for automatic pairing of volatile composition dispensers based on a spatial relationship. Automatic pairing functionality enables volatile composition dispensers to establish wireless communication when positioned within a predetermined proximity range. For example, and without limitation, the pairing process may activate when a separation distance of, for example, below ten meters, below five meters, below four meters, or below 3 meters is detected. The volatile composition dispensing system may use ultra-wideband transceivers to measure distances continuously and trigger pairing sequences when volatile composition dispensers are brought into close proximity during initial setup or reconfiguration operations.

Instead of or in addition to the heating element(s), a volatile composition dispenser may include an evaporative assistance element other than a heating element. The evaporative assistance element may be used to assist with the evaporation of a volatile composition from the evaporative surface. For example, the evaporative assistance element may be selected from the group consisting of a heater, a fan, an agitation member or agitator that cause vibration, both powered agitator and manual agitator, or combinations thereof. The evaporative assistance element may also include a chemical constituent to speed evaporation or evaporation rates, use of a chemically heated membrane to provide increased evaporation via exothermic reaction, or synergistic combinations thereof. The evaporative assistance element may also increase the amount of surface area of a delivery engine exposed to the evaporative assistance element, may cause a pressure gradient, rheostat, etc.

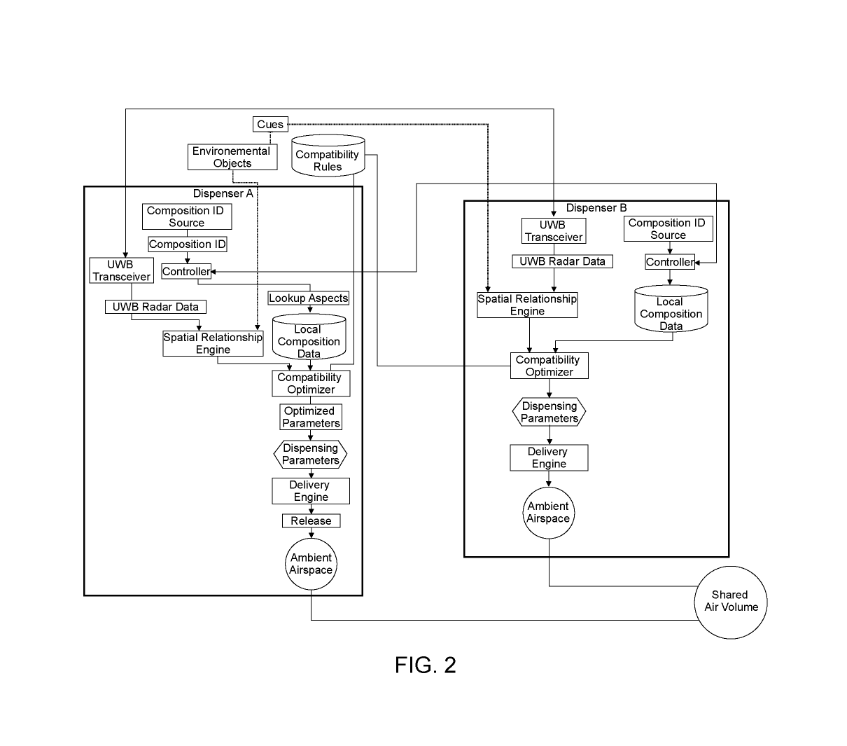

Referring to FIG. 2, a volatile composition dispensing system is illustrated that demonstrates the coordination of fragrance dispensing across multiple volatile composition dispensers. The volatile composition dispensing system may comprise Dispenser A and Dispenser B, each configured to process spatial data, compositional data, and compatibility data to optimize fragrance release for a desired olfactory experience.

Dispenser A may include a composition identification component (e.g., Composition ID Source) that provides identification data corresponding to volatile compositions housed within Dispenser A. The composition identification component may communicate with a UWB transceiver of Dispenser A that captures UWB radar data for spatial analysis. A controller of Dispenser A may receive composition identification data from the composition identification component and may determine operational and dispensing parameters for Dispenser A. The UWB transceiver may provide UWB radar data to a spatial relationship engine of Dispenser A that determines positioning information relative to other volatile composition dispensers and environmental objects. The spatial relationship engine may analyze time-of-flight calculations, signal strength variations, and reflection patterns to determine positioning information that may include distance measurements, angular orientations, and environmental obstacle detection.

The controller of Dispenser A may process local composition data that contains information regarding fragrance notes, volatility characteristics, boiling points, odor detection thresholds, and chemical properties of volatile compositions within Dispenser A. The controller may process the local composition data to coordinate dispensing parameters and communicate with other components of the volatile composition dispensing system.

A compatibility optimizer in Dispenser A may evaluate fragrance note combinations based on spatial relationship data from the spatial relationship engine, compositional aspects from the composition identification component, and compatibility rules retrieved from a compatibility rules database. The compatibility rules database may store compatibility matrices, fragrance interaction data, and optimization algorithms. The compatibility optimizer may generate compatibility scores that quantify how well different fragrance profiles blend when released in a shared environment. These compatibility scores may be based on fragrance chemistry principles, sensory harmony data, and volatility characteristics of individual fragrance components. The compatibility optimizer may process spatial constraints, reach feasibility parameters, and thermal activation requirements to determine optimal fragrance note selections and dispensing parameters. Dispensing parameters may include temperature settings, fragrance note activation patterns, intensity levels, and timing sequences that coordinate fragrance release across multiple volatile composition dispensers.

A delivery engine may receive commands based on the dispensing parameters and control the volatilization and release of volatile compositions into an ambient airspace surrounding Dispenser A. The delivery engine may include heating elements, evaporative assistance elements, and cartridge interfaces that enable selective activation of specific fragrance notes based on the optimized parameters. The delivery engine may output volatile compositions into the ambient airspace surrounding Dispenser A.

Dispenser B may include a composition identification component, a UWB transceiver, a controller, a spatial relationship engine, a compatibility optimizer, and a delivery engine. A composition identification component (e.g., Composition ID Source) in Dispenser B may provide composition identification data for volatile compositions housed within the Dispenser B. A UWB transceiver of Dispenser B may capture UWB radar data and communicate with the UWB transceiver of Dispenser A to establish spatial relationships between the two volatile composition dispensers. A controller may manage operations of Dispenser B and coordinate with the controller of Dispenser A through bidirectional communication.

The UWB transceiver of Dispenser B may provide UWB radar data to a spatial relationship engine of Dispenser B that may determine positioning information based on the UWB radar data. The controller of Dispenser B may access local composition data corresponding to volatile compositions in Dispenser B. A compatibility optimizer may receive spatial relationship data from the spatial relationship engine, local composition data from the composition identification component, and compatibility data from the compatibility rules database to generate optimized dispensing parameters for Dispenser B.

A delivery engine of Dispenser B may receive commands based on the optimized dispensing parameters and control the volatilization and release of volatile compositions into an ambient airspace surrounding Dispenser B. The delivery engine may include heating elements, evaporative assistance elements, and cartridge interfaces that enable selective activation of specific fragrance notes based on the optimized parameters. The delivery engine may output volatile compositions into the ambient airspace surrounding Dispenser B.

Environmental objects may represent physical barriers, furniture, walls, and other obstacles detected by the UWB transceivers during spatial analysis. The UWB transceiver of Dispenser A and UWB transceiver of Dispenser B may conduct continuous spatial sensing to monitor changes to the surrounding environment (e.g., new obstacle detection).

The ambient airspace outputs from both Dispenser A and Dispenser B may merge into a shared air volume where volatile compositions interact to create a coordinated olfactory experience. The volatile composition dispensing system may comprise real-time data exchange between volatile composition dispensers, continuous spatial monitoring, and adjustment of dispensing parameters to maintain optimal fragrance harmony across the shared air volume.

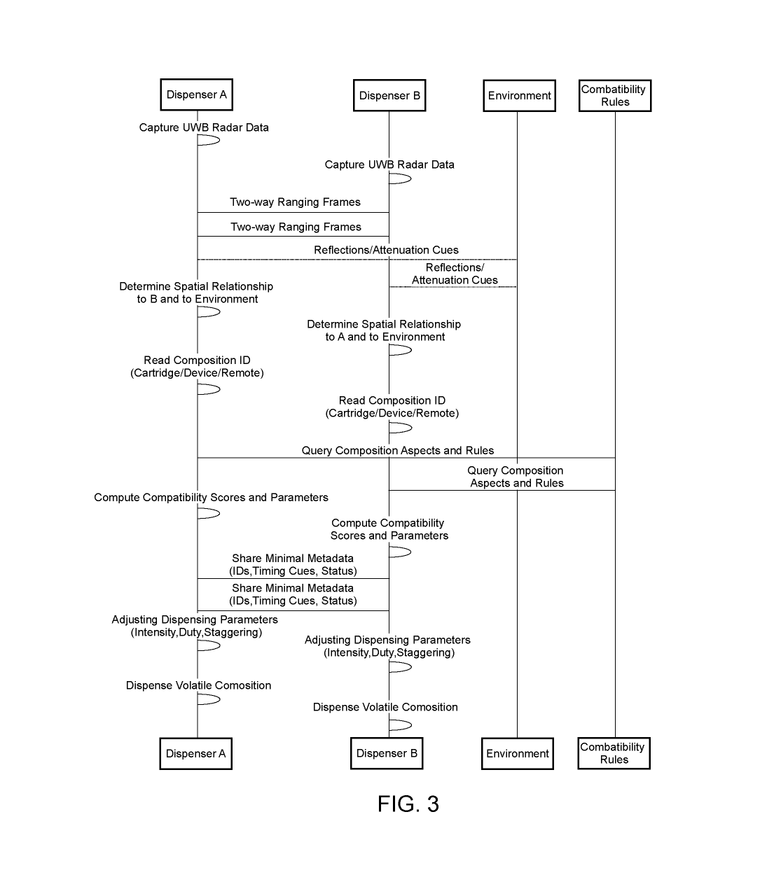

Referring to FIG. 3, optimizing fragrance dispensing across multiple volatile composition dispensers of a volatile composition dispensing system is illustrated. The volatile composition dispensing system may comprise parallel processing paths for multiple volatile composition dispensers. The volatile composition dispensing system may provide synchronized dispensing operations based on spatial relationships, compositional analysis, and fragrance compatibility assessments.

The volatile composition dispensing system may begin with both Dispenser A and Dispenser B independently capturing UWB radar data through respective ultra-wideband transceivers. This UWB radar data enables each volatile composition dispenser to gather spatial relationship information regarding its surrounding environment and the presence of other volatile composition dispensers.

Following the spatial relationship data capture, Dispenser A and Dispenser B may perform two-way ranging protocols to establish precise spatial measurements between the volatile composition dispensers. The two-way ranging protocols may comprise time-of-flight calculations and signal strength analysis to determine distance, angular orientation, and relative positioning between the volatile composition dispensers. This bidirectional communication enables both volatile composition dispensers to independently verify spatial relationship data and establish a coordinated volatile composition dispensing system.

The volatile composition dispensing system may process reflections and attenuation cues from the surrounding environment to identify physical barriers, obstacles, and spatial characteristics that can affect fragrance distribution. Environmental reflections detected by the UWB transceivers may provide information regarding walls, furniture, and other objects positioned between or near the volatile composition dispensers. Attenuation measurements may indicate the presence of materials that absorb or deflect radio frequency signals, which can correlate with barriers that affect fragrance diffusion.

Both Dispenser A and Dispenser B may determine a spatial relationship relative to each other and to a surrounding environment based on the UWB radar data and environmental reflection analysis. The spatial relationship determination may include distance measurements, angular positioning, obstacle detection, and zone identification.

Each volatile composition dispenser may receive and/or process composition identification data from cartridge identifiers, dispenser memory, or input from a remote device. Each volatile composition dispenser may determine composition identification data based on near-field communication tags, radio frequency identification systems, quick response codes, or manual user selection through a remote device interface. The composition identification data may enable each volatile composition dispenser to determine specific fragrance notes, volatility characteristics, and chemical properties of the volatile compositions available for dispensing.

The volatile composition dispensing system may query compositional aspects and compatibility rules from a compatibility rules database. This compatibility rules database may provide fragrance note profiles, compatibility matrices, volatility data, odor detection thresholds, and optimization algorithms. The compositional aspects may include information regarding individual fragrance compounds, thermal activation requirements, and fragrance interaction characteristics.

Both Dispenser A and Dispenser B may determine fragrance compatibility and optimize dispensing parameters based on the spatial relationship data, compositional aspects, and compatibility rules. The fragrance compatibility determination may evaluate potential fragrance note combinations, assess reach feasibility for a determined spatial configuration, and generate compatibility scores that quantify the blending potential of different fragrance note pairings. The dispensing parameter optimization may determine temperature settings, fragrance note activation patterns, dispensing intensity levels, and timing sequences that maximize compatibility scores for a determined spatial configuration.

The volatile composition dispensers may exchange metadata including device identifiers, timing cues, and operational status information to support coordinated fragrance release. This metadata exchange may enable synchronization of dispensing activities, coordination of fragrance note activation sequences, and real-time adjustment of dispensing parameters based on changing conditions or user preferences.

Both Dispenser A and Dispenser B may adjust their respective dispensing parameters based on the fragrance compatibility scores and shared metadata. The dispensing parameter adjustments may include modifications to temperature settings, intensity levels, and timing sequences that optimize the coordinated fragrance experience. The dispensing parameter adjustments may occur in response to spatial relationship changes, environmental condition variations, or user preference updates.

Dispenser A and Dispenser B may dispense their respective volatile compositions in a coordinated manner that creates a harmonized olfactory experience across the shared environment. The coordinated volatile composition dispensing may involve synchronized activation of compatible fragrance notes, staggered release patterns that create temporal variation, or zone-based dispensing strategies that optimize scent distribution based on spatial configuration and environmental characteristics.

### VOLATILE COMPOSITION DISPENSING SYSTEM ARCHITECTURE MODULES

A volatile composition dispensing system may include: (1) a spatial mapping module, (2) a fragrance compatibility module, (3) a parameter optimization module, (4) a control and scheduling module, (5) a user interface module, and (6) a sensing and feedback module. The spatial mapping module may provide location and environmental data to the fragrance compatibility module for spatial constraint evaluation. The fragrance compatibility module may provide fragrance compatibility assessments to the parameter optimization module and the control and scheduling module for determining dispensing parameters. The user interface module may receive status updates and provide user commands to the control and scheduling module. The sensing and feedback module may continuously provide environmental data to the parameter optimization module and the control and scheduling module for operational adjustments.

### SPATIAL MAPPING MODULE

The spatial mapping module may determine and monitor spatial relationships between volatile composition dispensers within an environment. The spatial analysis module may be configured to (1) determine separation distances between volatile composition dispensers, (2) determine angular relationship between volatile composition dispensers, and (3) identify and map environmental objects (e.g., physical barriers, furniture, walls) that can affect scent distribution patterns. The spatial analysis module can be configured to process ultra-wideband radar data and determine: (1) time difference of arrival (TDoA), (2) phase difference of arrival (PDoA), and (3) angle of arrival (AoA) measurements. The spatial analysis module may create environmental models that represent physical spaces including, but not limited to, two-dimensional floor plans, three-dimensional floor plans, or zone-based representations.

The spatial mapping module may calculate distances and directional relationships between volatile composition dispensers to determine a spatial relationship between volatile composition dispensers. These calculations may identify overlapping regions where fragrances may interact, and non-overlapping regions where fragrances do not interact. The spatial mapping module may continuously update spatial relationship data to accommodate changes in device positioning or environment configuration.

### FRAGRANCE COMPATIBILITY MODULE

A volatile composition dispensing system may use fragrance compatibility scoring to evaluate fragrance note combinations to determine harmonious fragrance note pairings for optimizing dispensing across multiple volatile compositions dispensers. A fragrance compatibility module may control which volatile compositions can interact without creating undesired olfactory experiences. The fragrance compatibility module may maintain fragrance profiles including fragrance note compositions, volatility characteristics, and intensity parameters for volatile compositions. The fragrance compatibility module may include a fragrance database. The fragrance database stores composition data, compatibility matrices, and chemical property information corresponding to volatile compositions. The fragrance compatibility module may include a scoring module configured to determine how different fragrance compositions interact when released by volatile composition dispensers. The scoring module can generate a compatibility score based on chemistry principles and sensory harmony data.

Compatibility scores may be generated that represent a quantitative measure of how well fragrance notes blend when released in a shared environment. The compatibility scores may be normalized on a scale from 0 to 1, where 0 indicates incompatible or clashing combinations that result in undesired olfactory experiences, and 1 represents highly compatible pairings that create harmonious blended fragrances.

For example, lavender and ylang-ylang may have a compatibility score of 0.9, indicating a highly compatible pairing based on the shared floral characteristics. For further example, lavender and eucalyptol may have a compatibility score of 0.4 due to the contrasting olfactory characteristics.

The compatibility scoring can be updated based on user feedback, additional sensory data, and analysis for new fragrance pairings. Further, the compatibility scoring may be affected based on location-specific requirements, user preferences (e.g., user-defined incompatibility lists), and safety considerations. The fragrance compatibility module may integrate these additional limitations with the spatial mapping data to determine which volatile compositions may be dispensed simultaneously across different locations in an environment.

### VOLATILITY AND REACH CHARACTERIZATION

The fragrance compatibility module may be configured to determine reach feasibility for a combination of fragrance notes. To determine reach feasibility, each fragrance note may be characterized by volatility characteristics including boiling point and odor detection threshold (ODT). High-volatility compounds (top notes) may have boiling points below about 200° C and diffuse relatively quickly, while low-volatility compounds (base notes) may have boiling points above about 250° C and require higher heat for effective diffusion. The odor detection threshold may define an amount of a given compound in air at which the compound becomes perceptible to a user. The volatility characteristics directly influence how far fragrance compounds can travel through air and remain detectable at various distances from the dispensing source.

The volatile composition dispensing system may generate a reach feasibility matrix Pᵢⱼ(D) for evaluating whether specific fragrance compounds can be perceived at designated distance D based on their volatility and odor threshold characteristics. The binary matrix assigns values of 1 to indicate that compounds are perceivable at the specified distance, while values of 0 indicate that compounds cannot be detected reliably at that distance due to insufficient volatility or excessive odor threshold requirements.

The reach feasibility matrix may include both volatility characteristics and odor detection thresholds to determine effective diffusion ranges for each fragrance compound. High-volatility compounds with low odor thresholds receive binary values of 1 for both close-range distances (e.g., 3-5 meters) and far-range distances (e.g., 10-50 meters), indicating detectability across the full operational range. Moderate-volatility compounds may receive binary values of 1 for close-range applications but 0 for far-range distances where airborne concentrations fall below detection thresholds. Low-volatility compounds such as coumarin typically receive binary values of 1 for close-range distances where limited diffusion still enables detection, but 0 for far-range distances where insufficient volatilization prevents adequate airborne concentrations.

The reach feasibility matrix enables the volatile composition dispensing system to select appropriate fragrance combinations based on spatial requirements and ensure that selected fragrance compounds can achieve perceivable concentrations at the intended operational distances.

Suitable volatile compositions are described in U.S. Patent No. 12,036,341 and U.S. Patent Application No. 18/229,043, each of which is hereby incorporated herein by reference in its entirety, and U.S. Provisional Patent Application No. 63/892,897.

### PARAMETER OPTIMIZATION MODULE

The parameter optimization module may receive data from the spatial analysis module and the fragrance compatibility module to determine optimal dispensing parameters for coordinating fragrance release. The parameter optimization module may comprise an optimization algorithm that determines fragrance note activation patterns and temperature settings to maximize compatibility scores across multiple volatile composition dispensing devices while satisfying spatial, thermal, and compositional constraints. The parameter optimization module can process spatial relationship data, fragrance compatibility scores, and device capabilities to generate coordinated dispensing parameters that create harmonious scent experiences across different environmental configurations.

Without wishing to be bound by theory, the optimization algorithm may maximize an objective function expressed mathematically as:

### ∑₍i ∈ A₎ ∑₍j ∈ B₎ Cᵢⱼ· xᵢ yⱼ Pᵢⱼ(D)

where the summation encompasses all possible fragrance note pairs between devices positioned at distance D. The variable Cᵢⱼ represents the compatibility score between fragrance note i from a first volatile composition dispenser (Device A) and fragrance note j from a second volatile composition dispenser (Device B), with values ranging from 0 to 1. The binary variables xᵢ and yⱼ indicate activation states for individual fragrance notes, where values of 1 represent activated notes that are volatilized and released into the environment, while values of 0 represent inactive notes that remain in non-volatile states within their respective cartridges.

The reach feasibility parameter Pij(D) may provide a binary constraint that ensures both fragrance notes in each evaluated pair can achieve perceivable concentrations at the measured distance D between volatile composition dispensing devices. The optimization algorithm may only consider fragrance note combinations where both fragrance compounds possess sufficient volatility and low enough odor detection thresholds to create detectable airborne concentrations at the operational distance.

The parameter optimization module may process spatial relationship data from ultra-wideband transceivers to determine device separation distances, angular orientations, and environmental obstacles that influence fragrance diffusion patterns. Distance measurements enable selection of appropriate reach feasibility parameters Pᵢⱼ(D) for each potential fragrance note combination, while obstacle detection data may modify reach calculations to account for walls, furniture, or other barriers that impede scent distribution between dispensers.

The parameter optimization module may analyze cartridge identifier information to determine available fragrance notes within each volatile composition dispenser and associated chemical properties including volatility characteristics, odor detection thresholds, and compatibility relationships with notes in other devices. The parameter optimization module may access fragrance databases that contain detailed compositional information for each cartridge type, enabling informed selection decisions based on the specific compounds available for activation.

The parameter optimization module retrieves compatibility scores Cᵢⱼ for each potential fragrance pairing and incorporates these values into the objective function calculation to prioritize combinations that produce harmonious blended experiences. Higher compatibility scores receive greater weight in the optimization calculation, increasing the likelihood that harmonious combinations will be selected over potentially clashing alternatives.

Real-time optimization capabilities enable dynamic adjustment of fragrance note activation patterns and temperature settings in response to changing spatial relationships, environmental conditions, or user preferences. The parameter optimization module may continuously monitor ultra-wideband radar data to detect device movement and automatically recalculate optimal dispensing parameters when spatial configurations change. Environmental sensors may provide additional input data including ambient temperature, humidity, and air circulation patterns that influence fragrance diffusion characteristics and optimization calculations. Further, information from other locally connected devices (e.g., a connected fan or thermostat) may be used to adjust the dispensing parameters.

### DISPENSING PARAMETER CONTROL AND ADJUSTMENT

The parameter optimization module may adjust multiple dispensing parameters to optimize fragrance release based on spatial relationships, compatibility scores, and environmental configurations. These dispensing parameters include but are not limited to temperature settings, fragrance note activation patterns, and dispensing intensity characteristics that determine the olfactory experience created by coordinated volatile composition dispensers. The parameter optimization module may adjust heating settings for one or more volatile composition dispensers to selectively volatilize compounds based on boiling points, volatility characteristics, and a spatial configuration between volatile composition dispensers. In examples where a non-heater evaporative assistance element is present (e.g., a fan), the optimization module may adjust the related dispensing settings (e.g., speed of the fan). The parameter optimization module may control which fragrance notes are activated or deactivated within a volatile composition dispenser based on compatibility assessments, spatial relationships, and desired olfactory outcomes. The parameter optimization module can further control dispensing intensity, dispensing duration, and dispensing timing parameters to optimize scent distribution.

The parameter optimization module may generate temperature setting recommendations for each volatile composition dispenser based on the volatility requirements of selected fragrance notes and the desired intensity levels for the coordinated scent experience. Temperature optimization may involve selecting minimum temperatures that enable adequate volatilization of all activated notes, or higher temperatures that enhance projection and diffusion characteristics for extended-range applications. Multi-zone temperature control may be implemented in devices with multiple heating elements, enabling selective activation of different fragrance notes through spatially distributed thermal management.

Temperature settings may control volatilization rates and diffusion characteristics of fragrance notes. The volatile composition dispensing system may operate across a temperature range that ranges between room temperature, typically 20-25° C, to 120° C. This temperature range enables selective volatilization of compounds with different boiling points and thermal activation requirements, allowing the parameter optimization module to activate specific fragrance notes while maintaining others in non-volatile states within the cartridge reservoir.

The parameter optimization module may select temperature settings based on volatility characteristics of activated fragrance notes and the spatial configuration of volatile composition dispensers. For example, in close-distance environments with device separation between about 3 to 5 meters, the temperature range may be set to between 50-70° C to create intimate scent experiences. For further example, in far-distance environments, the temperature range may be set to between about 100-120° C. The higher temperatures enable volatilization of compounds with boiling points up to 250° C or higher, ensuring that sufficient vapor concentrations are generated to maintain perceivable fragrance levels at the intended operational distances. The elevated temperature settings also increase the volatilization rates of lower-boiling compounds, enhancing their diffusion characteristics and enabling effective scent blending across large environmental spaces.

The parameter optimization module may initiate heating at lower temperatures to volatilize high-volatility compounds first, then gradually increase temperature to activate moderate and low-volatility compounds in sequence. The sequential activation prevents rapid depletion of high-volatility fragrance notes.

Fragrance note activation and deactivation represent selective dispensing parameters that determine which specific fragrance notes within multi-component cartridges are volatilized during coordinated dispensing operations. Rather than dispensing all compounds uniformly, the parameter optimization module may activate only those fragrance notes that optimize compatibility scores and satisfy spatial reach requirements for the current device configuration. This selective activation capability enables fragrance customization based on optimization algorithm results and changing environmental conditions.

The activation process may involve applying thermal energy selectively to cartridge regions containing specific fragrance compounds, or utilizing temperature profiles that preferentially volatilize compounds with particular boiling point ranges. Deactivation of fragrance notes may involve reducing thermal energy to levels below the effective volatilization temperature for specific compounds, preventing their release into the environment while maintaining activation of other selected notes. The deactivation process may be implemented gradually to avoid abrupt changes in fragrance character, or rapidly when spatial relationships change and different note combinations become optimal for the new configuration.

Dispensing intensity parameters control the rate and concentration of fragrance compound release into the environment. Intensity adjustments may be achieved through temperature modulation, where higher temperatures increase volatilization rates and create stronger fragrance concentrations, while lower temperatures reduce release rates for more subtle scent experiences.

Dispensing duration parameters determine the temporal characteristics of fragrance release, including continuous operation, intermittent pulsing, or scheduled activation patterns. Continuous operation maintains steady fragrance concentrations throughout an operational period, while intermittent pulsing may create varying intensity levels that prevent olfactory adaptation and maintain user awareness of the fragrance presence. Scheduled activation enables coordination of fragrance release with specific time periods, user presence detection, or environmental conditions.

The parameter optimization module may adjust dispensing parameters dynamically in response to changing spatial relationships detected through ultra-wideband ranging measurements. When volatile composition dispensers are moved closer together, the controller may reduce temperature settings and select different fragrance note combinations optimized for close-range compatibility. Conversely, when volatile composition dispensers are separated to greater distances, the controller increases temperature settings and activates high-volatility compounds with strong projection characteristics to maintain effective scent coordination across the expanded spatial configuration.

Dispensing parameters may be adjusted based on compatibility scores. Fragrance note combinations that achieve higher compatibility scores while satisfying spatial and thermal constraints may be prioritized. The parameter optimization module may adjust temperature settings to enable activation of highly compatible note pairs even when some compounds require elevated volatilization temperatures. Intensity parameters may be balanced between devices to ensure that compatible fragrance notes blend harmoniously without one volatile composition dispenser overwhelming the contributions from other volatile composition dispensers.

The parameter optimization module may adjust dispensing parameters based on environmental objects (e.g., obstacles, air circulation patterns). The parameter optimization module may increase temperature settings and dispensing intensity when obstacles are detected between volatile composition dispensers, compensating for reduced diffusion efficiency caused by physical barriers.

The parameter optimization module may store preference profiles that modify optimization calculations to favor specific fragrance note categories, intensity levels, or activation patterns preferred by users. The preference profiles may include preferences for fragrance intensity, note selection, and temporal characteristics.

Power management considerations may influence dispensing parameter adjustment to balance fragrance performance with energy consumption requirements. The parameter optimization module may implement power-efficient temperature profiles that achieve adequate volatilization while minimizing heating element energy consumption. Duty cycle modulation may alternate between active heating periods and passive cooling periods to maintain average temperature levels while reducing overall power consumption. Example emission cycles are described in U.S. Patent No. 11,896,743, which is hereby incorporated herein by reference in its entirety, and U.S. Provisional Patent Application No. 63/892,897.

### VISUAL INDICATOR CONTROL

The parameter optimization module may control operations of visual indicators of volatile composition dispensers. The visual indicators of volatile composition dispensers may comprise light-emitting diodes (LEDs), organic light-emitting diodes (OLEDs), or other illumination technologies that provide color, intensity, and pattern control capabilities. Visual indicators may display system status information including power status, fragrance levels, connectivity status, and operational modes through various color codes and lighting patterns. Additionally, or alternatively, visual indicators may provide ambient or mood lighting effects.

The parameter optimization module can coordinate visual indicators between multiple volatile composition dispensers to provide synchronized lighting effects that complement coordinated fragrance dispensing. When multiple devices are operating in coordination, the visual indicators may display synchronized color patterns, intensity variations, or sequential lighting effects that visually represent the coordinated fragrance experience. The synchronized lighting effects may enhance the overall sensory experience by providing visual cues that correspond to fragrance transitions, intensity changes, zone-based fragrance programs, or user-designated lighting effects, etc.

The parameter optimization module may provide lighting effects that indicate the distance and coordination status between multiple dispensers within an environment. Close-range spatial relationships may trigger synchronized color matching between synchronized volatile composition dispensers, creating visual confirmation that the dispensers are operating as a coordinated system. Far-range relationships may provide complementary color schemes that indicate successful coordination while acknowledging the extended spatial separation between devices.

The adjustment to lighting characteristics may include static lighting effects where multiple devices display consistent colors or patterns, or dynamic lighting effects that change over time in synchronized patterns. Dynamic effects may include color transitions that correspond to fragrance note evolution, intensity pulsing that matches fragrance release cycles, or directional lighting patterns that indicate fragrance flow or zone transitions.

### CONTROL AND SCHEDULING MODULE

A control and scheduling module may manage timing, intensity, and sequencing of fragrance dispensing across multiple volatile composition dispensers. The control and scheduling module may include input from the spatial mapping module, the fragrance compatibility module, and the parameter optimization module to generate coordinated dispensing commands for individual volatile composition dispensers.

The control and scheduling module may include zone-based control where different compatible fragrance notes are assigned to specific areas, path-based control to manage fragrance transitions along user movement routes, and time-varying settings that layer fragrances harmoniously a temporal sequence. The control and scheduling module may adjust dispensing intensity based on spatial relationships, occupancy detection, and environmental conditions.

### USER INTERFACE MODULE

The volatile composition dispensing system may include a user interface that may display detected volatile composition dispensers, spatial relationships of volatile composition dispensers, and a pairing status. Users may select specific volatile composition dispensers for pairing or unpairing operations, configured distance threshold for automatic pairing, and establish preferences for olfactory experiences.

A user interface module may provide control and monitoring of the volatile composition dispensing system via a remote device. The user interface module may include monitoring dashboards that show active fragrance notes per volatile composition dispensers, estimated fragrance concentration by zone, and system status information. The user interface module may enable automatic pairing and unpairing of volatile composition dispensers based on proximity detection and user preferences. When volatile composition dispensers are positioned within a predetermined proximity threshold, the user interface module may prompt users to establish a pairing relationship through an application interface. The user interface module may automatically unpair volatile composition dispensers when one volatile composition dispenser is moved beyond a predetermined distance limit, allowing for dynamic reconfiguration of a volatile composition dispensing system. The user interface may allow the user to determine a distance from the remote device to a volatile composition dispenser. The user interface may help guide a user towards the volatile composition dispenser, which may be helpful in locating devices (e.g., if a portable device was moved).

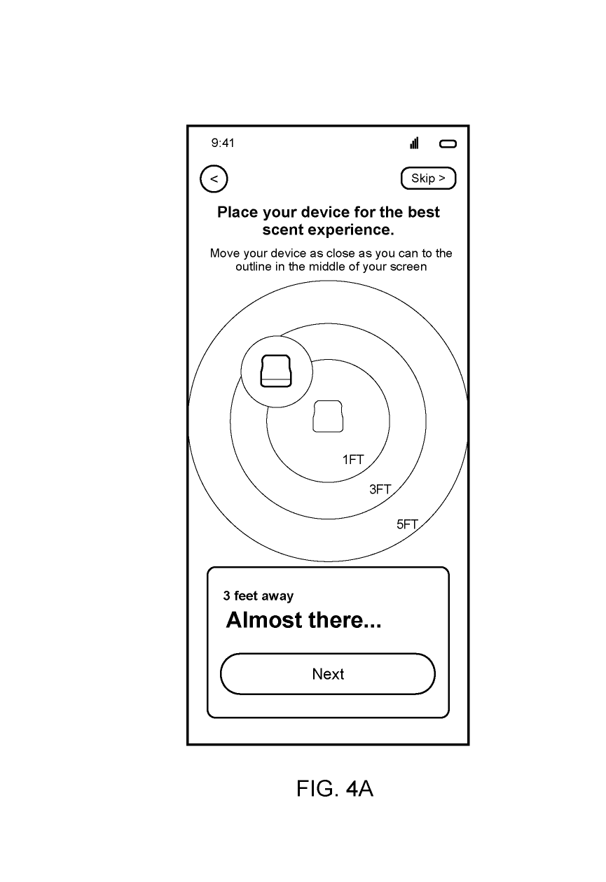

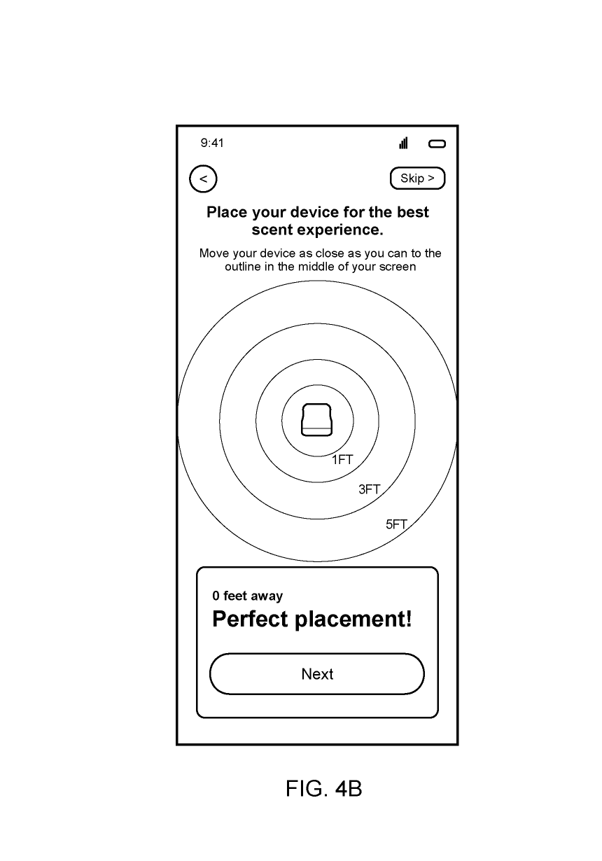

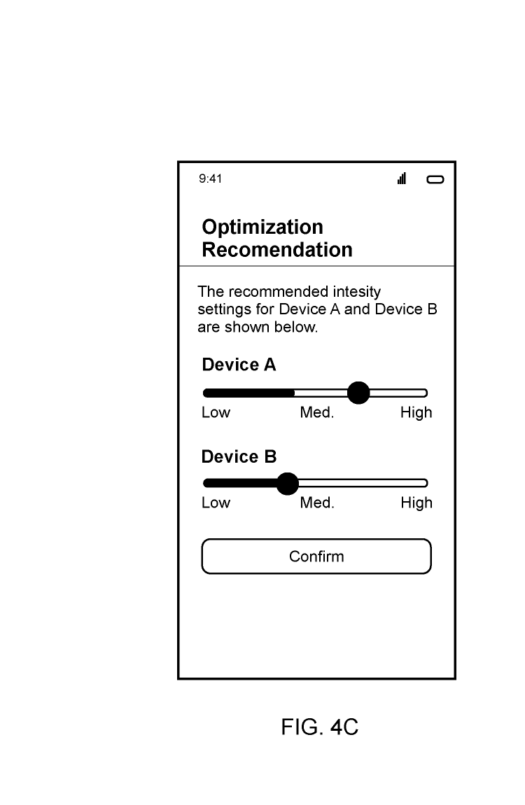

In reference to FIGS. 4A and 4B, the volatile composition dispensing system may comprise a software application that may be displayed via a user interface. The volatile composition dispensing system may include a dashboard corresponding to the positioning of a volatile composition dispenser. The volatile composition dispensing system may generate a visual representation of the positioning of a volatile composition dispenser. The visual representation may indicate whether the volatile composition dispenser is in an optimal location based on a desired olfactory experience. If the volatile composition dispenser is not in an optimal location, the volatile composition dispensing system can generate an alert and a recommendation corresponding to the optimal location.

In reference to FIG. 4C, the volatile composition dispensing system may include a dashboard corresponding to the intensity settings of one or more volatile composition dispensers in a volatile composition dispensing system. The volatile composition dispensing system may generate a visual representation of the intensity setting of a volatile composition setting and a corresponding recommendation for the intensity level. The volatile composition dispensing system may include an adjustable interactive component (e.g., slider) via the user interface. A user may adjust the intensity of a volatile composition dispenser by changing the positioning of the adjustable interactive component.

### SENSING AND FEEDBACK MODULE

A sensing and feedback module may perform presence detection, environmental monitoring and concentration estimation to support adaptive fragrance dispensing. The sensing and feedback module may include external sensors including, but not limited to, motion sensors, occupancy detectors, temperature and humidity sensors, airflow sensors, and volatile organic compound sensors. The sensing and feedback module may determine occupancy patterns, environmental conditions, and estimated fragrance concentrations for a corresponding environment based on sensor data. Example environmental conditions and uses thereof are described in U.S. Patent Application No. 18/229,043. The sensing and feedback module may incorporate information from other locally connected devices (e.g., a connected doorbell or thermostat).

The sensing and feedback module may provide input to the parameter optimization module and/or the control and scheduling module for adjustment of dispensing parameters based on real-time conditions. For example, when user occupancy is detected in specific zones, the sensing and feedback module may increase fragrance intensity or activate dormant volatile composition dispensers. When an area becomes unoccupied, the sensing and feedback module may reduce dispensing intensity or stop fragrance release.

### COMPATIBILITY EXAMPLE

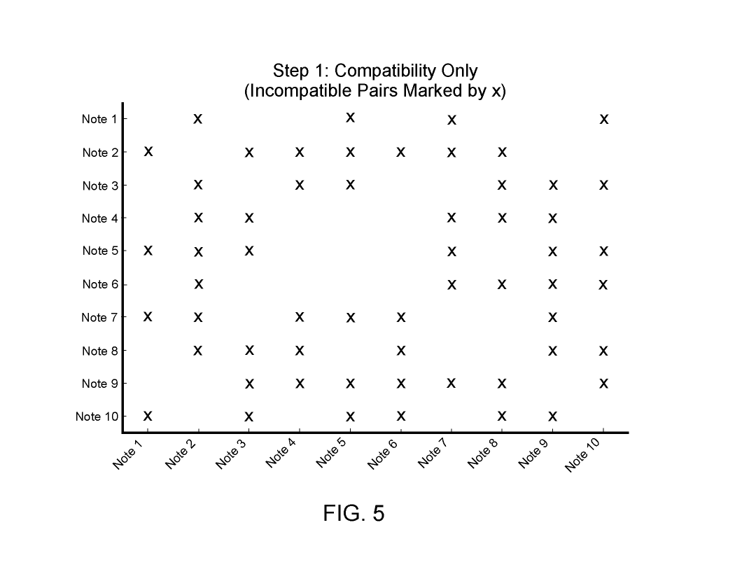

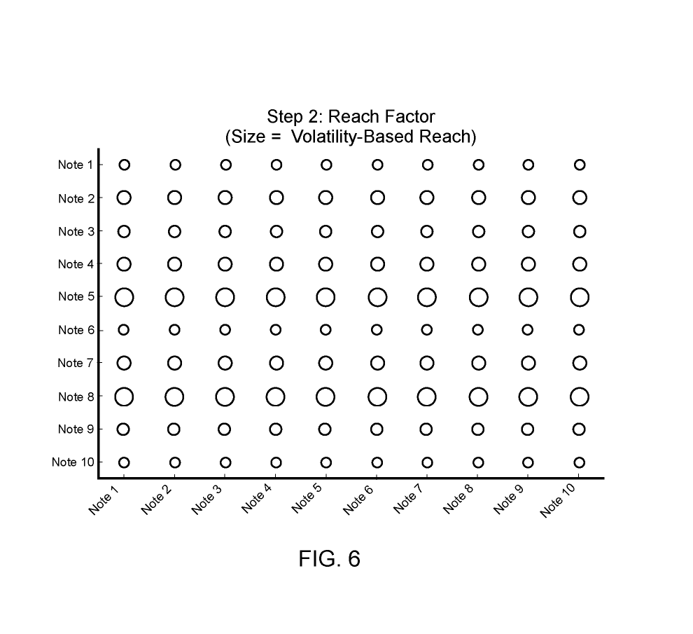

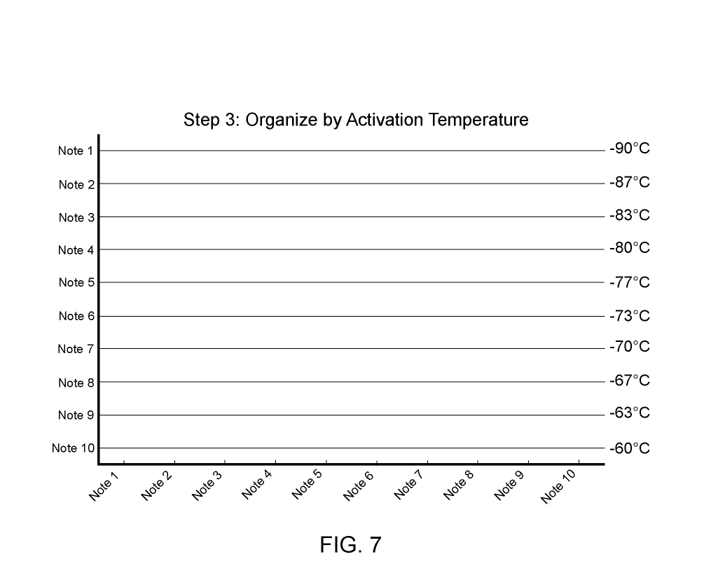

Referring to FIG. 5, the volatile composition dispensing systems may include determining a compatibility between fragrance notes of volatile compositions of different volatile composition dispensers in a volatile composition dispensing system. As illustrated in FIG. 6, the volatile composition dispensing system may further include determining a reach factor for each fragrance note in a volatile composition of a volatile composition dispenser. The size of a circle corresponds to the distance that a fragrance note will travel. The volatile composition dispensing system, as shown in FIG. 7, may organize fragrance notes by activation temperature.

The volatile composition dispensing system may include two volatile composition dispensers. The volatile composition dispensers may be in wireless communication via a network. Each volatile composition dispenser can be configured for near-field communication (NFC) identification of a corresponding volatile composition, ultra-wideband proximity, angle of arrival, and boundary detection.

Via the NFC identification, the volatile composition dispensing system may determine similarities and differences between volatile compositions of volatile composition dispensers in the volatile composition dispensing system. The volatile composition dispensing system may determine, via the UWB boundary detection, a proximity of an environmental object (e.g., wall) and a spatial relationship between each volatile composition dispenser. The spatial relationship may indicate where volatile compositions dispensers are in the same room and the distance between the volatile composition dispensers. The volatile composition dispensing system may be able to determine when volatile compositions dispensers are in different rooms.

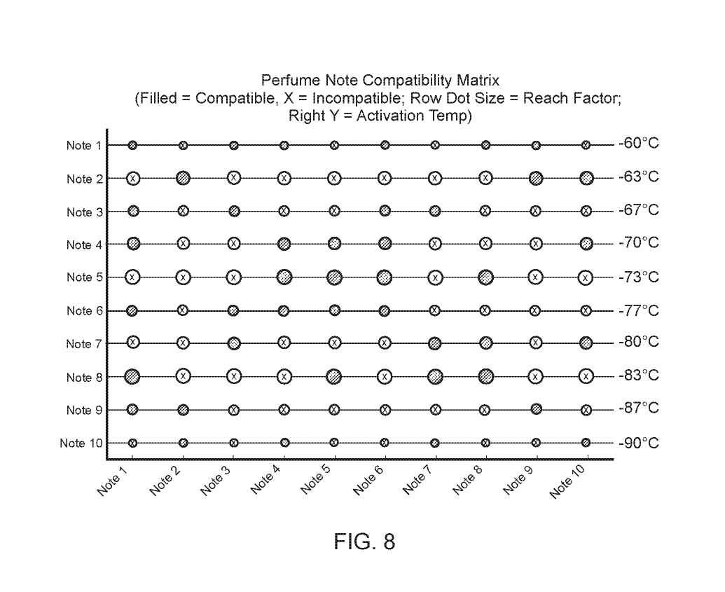

In reference to FIG. 8, the volatile composition dispensing system may be configured to create a perfume or fragrance note compatibility matrix. The perfume note compatibility matrix may indicate whether fragrance notes are compatible, a reach factor for a fragrance note combination, and an activation temperature corresponding to a fragrance note combination.

The volatile composition dispensing system may maximize the sum of compatibility scores across fragrance note pairs that are actively selected and detectable at the separation distance of volatile composition dispensers. The volatile composition dispensing system can determine one or more fragrance note pairs that are optimal for a desired olfactory experience based on the fragrance note compatibility, and the reach factor.

### EXAMPLES

As illustrated below, a volatile composition dispensing system can be configured for coordinating the release of fragrance notes across multiple volatile composition dispensing devices based on spatial configuration and thermal control. The volatile composition dispensing system may integrate odor chemistry (e.g., threshold, volatility) with sensory compatibility data to select optimal fragrance note pairings for close environments and far environments. For example, and without limitation, the volatile composition dispensing system may select a fragrance note pairing to create a warm, subtle, personal olfactory experience for close embodiments and a bold, clean, space-filling olfactory experience for far environments.

### CLOSE-RANGE EXAMPLE

The volatile composition dispensing system may optimize fragrance notes for different spatial configurations, with close-range scenarios encompassing device separations between 3 meters and 5 meters, and far-range scenarios covering separations between 10 meters and 50 meters. The volatile composition dispensing system may use different fragrance note selection criteria, temperature profiles, and dispensing parameters to create desired olfactory experiences for each spatial configuration.

Close-range operational scenarios prioritize subtle, intimate scent experiences that avoid overwhelming users positioned near the volatile composition dispensers. For close-range configurations, fragrance notes with moderate volatility characteristics and limited diffusion ranges are prioritized to create localized scent zones without excessive projection beyond the immediate vicinity of the devices. Heating settings may range from 50° C to 70° C, providing sufficient thermal energy to volatilize selected compounds while preventing rapid depletion of fragrance materials and avoiding overpowering scent concentrations.

The fragrance note selection process for close-range scenarios may emphasize compounds with boiling points between 200° C and 300° C, that achieve adequate volatilization at moderate heating temperatures while maintaining controlled diffusion characteristics. These compounds provide sufficient olfactory presence for users within the 3-5 meter operational range while avoiding excessive projection that might create unpleasant scent concentrations or interfere with activities in adjacent areas.

For illustrative purposes, a volatile composition dispensing system may include two volatile composition dispensers with a separation distance between about 3 meters and about 5 meters. The volatile composition dispensing system is configured to activate or suppress fragrance notes based on a desired olfactory experience.

Each volatile composition dispenser includes lavender, coumarin, limonene, and linalool. The volatile composition dispensing system may be configured to create a subtle, blended olfactory environment near the volatile composition dispensers and limit activated fragrance notes to fragrance notes that are compatible at low diffusion ranges.

Lavender oil and coumarin may be selectively activated from a first volatile composition dispenser, while limonene and linalool are kept in an inactive state. Lavender oil can provide a soft herbal-floral note. Coumarin may provide an almond-vanilla sweetness. Lavender oil may be activated through controlled heating at temperature settings ranging between 50 to 60° C, allowing coumarin to slowly sublimate. The controlled heating at 50-60° C may provide gradual volatilization that maintains consistent fragrance output without creating overwhelming intensity levels that would be inappropriate for intimate spatial configurations.

A second volatile composition dispenser may include ylang-ylang and sweet orange. Ylang-Ylang can provide a warm floral note. The second volatile composition dispenser may heat the volatile composition between 60-70° C to release ylang-ylang mid notes.

Temperature coordination between the first volatile composition dispenser and the second volatile composition dispenser may optimize the release timing and intensity balance of the selected fragrance notes. Operation of the first volatile composition dispenser at 50-60° C may provide gentle lavender release while enabling passive coumarin sublimation, creating a foundation of herbal-floral and vanilla-almond notes. Operation of the second volatile composition dispenser at 60-70° C may provide slightly more active ylang-ylang release while maintaining minimal sweet orange diffusion, contributing warm floral and bright citrus accents to the established base.

The resulting fragrance experience may comprise a warm, intimate floral-spice blend that combines herbal lavender, vanilla-almond coumarin, warm ylang-ylang florals, and bright sweet orange accents in a harmonious composition. This blend may remain perceptible within a few meters of the volatile composition dispensers while avoiding excessive projection that would affect distant areas or create overwhelming intensity levels. The close-range optimization may enable users to experience a rich, layered fragrance that enhances the immediate environment without dominating the broader space.

The selective activation approach may demonstrate the capability of volatile composition dispensers to utilize subsets of available fragrance compounds based on spatial and compatibility optimization requirements. Each volatile composition dispenser may contain a plurality of fragrance notes within a cartridge while selecting compatible fragrance notes for specific configuration. This selective activation capability may enable adaptation to changing spatial configurations and user preferences without requiring physical cartridge replacement.

### FAR-RANGE EXAMPLE

Far-range environments require enhanced projection and diffusion capabilities to achieve perceivable fragrance concentrations across extended spatial separations between 10 meters and 50 meters. Far-range environments may prioritize fragrance notes with high volatility characteristics, low odor detection thresholds, and strong diffusion potential that enable effective scent coordination across large environmental spaces. Temperature control strategies for far-range environments employ elevated heating settings typically ranging from 100° C to 120° C to maximize volatilization rates and enhance projection characteristics of selected compounds.

The fragrance note selection process for far-range scenarios may target fragrance notes with boiling points below 200° C and odor detection thresholds below 0.02 parts per million, ensuring that selected fragrance notes achieve rapid volatilization and maintain perceivable concentrations at extended distances. These selection criteria favor top note compounds and highly volatile middle notes that possess the diffusion characteristics necessary for effective scent blending across large spatial separations.

For illustrative purposes, a volatile composition dispensing system may include two volatile composition dispensers with a separation distance between about 10 meters and about 50 meters. The volatile composition dispensing system is configured to activate or suppress fragrance notes based on a desired olfactory experience.

A first volatile composition dispenser may include lavender oil, coumarin, limonene, and linalool. The volatile composition dispensing system may selectively activate limonene and linalool while suppressing lavender oil and coumarin to prioritize high-volatility fragrance notes with desired projection characteristics for a far-range environment.

Limonene activation may occur through controlled heating at temperature settings between 100-110° C. Linalool activation may occur through controlled heating at 100-110° C. Linalool may contribute sweet lavender-like tones that complement the bright citrus characteristics of limonene while maintaining good projection properties for far-range applications.

A second volatile composition dispenser may contain ylang-ylang, sweet orange, eucalyptol, and citral fragrance notes. The volatile composition dispensing system may selectively activate eucalyptol and citral while maintaining ylang-ylang and sweet orange in inactive states. Eucalyptol may be activated through controlled heating between 110-120° C. Citral activation may occur through controlled heating at a range between about 110-120° C.

The combination of limonene's bright citrus top notes, linalool's soft floral-herbal middle notes, eucalyptol's fresh minty-camphorous characteristics, and citral's penetrating lemony-aldehyde notes produces a complex, harmonious fragrance profile that demonstrates successful coordination across far-range environments for producing a desired olfactory experience.

### COMBINATIONS

### EXAMPLE VOLATILE COMPOSITION DISPENSER EMBODIMENTS

A. A volatile composition dispenser comprising:
   a. a transceiver; and
   b. a controller, wherein the controller is configured for:
      i. determining a spatial relationship of the volatile composition dispenser relative to at least one of a surrounding environment and a second volatile composition dispenser; and
      ii. modifying at least one dispensing parameter based on the spatial relationship;
      iii. wherein the volatile composition dispenser is configured to dispense at least one volatile composition based on the at least one dispensing parameter.
B. The volatile composition according to Paragraph A, wherein the transceiver comprises an ultra-wideband transceiver configured to transmit and receive ultra-wideband radar data, wherein the spatial relationship is determined based on the ultra-wideband radar data.
C. The volatile composition according to Paragraph A or B, wherein the volatile composition dispenser comprises a cartridge, wherein the cartridge comprises a unique identifier corresponding to a respective composition of the at least one volatile composition, wherein the controller is configured to identify fragrance notes in the respective composition based on the unique identifier.
D. The volatile composition dispenser according to any of Paragraphs A to C, wherein the controller is configured to adjust the at least one dispensing parameter based on identified fragrance notes in the at least one volatile composition.
E. The volatile composition dispenser according to any of Paragraphs A, B, or D, wherein the controller is in operable communication with a remote device and configured to receive a unique identifier corresponding to a respective composition of the at least one volatile composition from the remote device.
F. The volatile composition dispenser according to any of Paragraphs A to E, wherein the controller is further configured for modifying the at least one dispensing parameter based on a volatile composition housed and dispensed by the second volatile composition dispenser.
G. The volatile composition dispenser according to any of Paragraphs A to F, wherein the surrounding environment comprises distance data and directional data related to at least one object in a proximity of the volatile composition dispenser.
H. The volatile composition dispenser according to any of Paragraphs A to G, wherein the volatile composition dispenser further comprises at least one visual indicator, wherein the controller is configured to adjust at least one lighting characteristic of the at least one visual indicator based on at least one of the spatial relationship and compositional aspects of the at least one volatile composition.

### EXAMPLE VOLATILE COMPOSITION DISPENSER EMBODIMENTS

A. A volatile composition dispensing system comprising:
   a. a first volatile composition dispenser comprising a first volatile composition, a first ultra-wideband transceiver, and a first controller;
   b. a second volatile composition dispenser comprising a second volatile composition, a second ultra-wideband transceiver, and a second controller;
      i. wherein the first controller is configured for modifying at least one dispensing parameter of the first volatile composition dispenser based on at least one of:
         1. a spatial relationship between the first volatile composition dispenser relative to the second volatile composition dispenser;
         2. compositional aspects of the second volatile composition;
         3. a compatibility score of the first volatile composition and the second volatile composition; and
         4. a unique identifier corresponding to the first volatile composition that is wirelessly communicated to the first controller.
B. The volatile composition dispensing system according to Paragraph A, wherein the second controller is configured for modifying at least one dispensing parameter of the second volatile composition dispenser based on at least one of the spatial relationship between the first volatile composition dispenser and the second volatile composition dispenser, compositional aspects of the first volatile composition, and the compatibility score of the first volatile composition and the second volatile composition.
C. The volatile composition dispensing system according to any of Paragraphs A or B, wherein the first controller is configured to adjust the at least one dispensing parameter based on at least two of the spatial relationship between the first volatile composition dispenser and the second volatile composition dispenser, compositional aspects of the second volatile composition, the compatibility score of the first volatile composition and the second volatile composition, and the unique identifier corresponding to the first volatile composition.
D. The volatile composition dispensing system according to any of Paragraphs A to C, wherein the first controller is configured to adjust the at least one dispensing parameter based on at least three of the spatial relationship between the first volatile composition dispenser and the second volatile composition dispenser, compositional aspects of the second volatile composition, the compatibility score of the first volatile composition and the second volatile composition, and the unique identifier corresponding to the first volatile composition.
E. The volatile composition dispensing system according to any of Paragraphs A to D, wherein the at least one dispensing parameter comprises at least one of a temperature setting, an activation of a fragrance note, and a deactivation of a fragrance note.
F. The volatile composition dispensing system according to any of Paragraphs A to E, wherein the spatial relationship comprises a distance between the first volatile composition dispenser and the second volatile composition dispenser, wherein the distance is between about 3 meters and about 50 meters.
G. The volatile composition dispensing system of according to any of Paragraphs A to F, wherein the first controller operates the first volatile composition dispenser at a first temperature range, wherein the second controller operates the second volatile composition dispenser at a second temperature range different from the first temperature range, wherein the first temperature range and the second temperature range are based on at least one of the spatial relationship and the compatibility score of the first volatile composition and the second volatile composition.
H. The volatile composition dispensing system of according to any of Paragraphs A to G, wherein the spatial relationship comprises distance data and directional data related to an object positioned between the first volatile composition dispenser and the second volatile composition dispenser.
I. The volatile composition dispensing system of according to any of Paragraphs A to H, wherein the first volatile composition dispenser comprises a cartridge, wherein the cartridge comprises the unique identifier corresponding to the first volatile composition, wherein the first controller is configured to identify fragrance notes of the first volatile composition based on the unique identifier.
J. The volatile composition dispensing system according to any of Paragraphs A to I, wherein the first volatile composition dispenser further comprises at least one visual indicator, wherein the first controller is configured to adjust at least one lighting characteristic of the at least one visual indicator based on at least one of the spatial relationship, compositional aspects of the second volatile composition, the compatibility score of the first volatile composition and the second volatile composition, and the unique identifier corresponding to the first volatile composition.
K. The volatile composition dispensing system according to Paragraph J, wherein the at least one lighting characteristic includes at least one of color, intensity, and direction.

### EXAMPLE VOLATILE COMPOSITION DISPENSING METHODS

A. A method of volatile composition dispensing, the method comprising:
   a. capturing, via an ultra-wideband transceiver of a first volatile composition dispenser, ultra-wideband radar data;
   b. determining, via a controller operably connected to the ultra-wideband transceiver, a spatial relationship between the first volatile composition dispenser and a second volatile composition dispenser, wherein the spatial relationship is determined based on the ultra-wideband radar data;
   c. determining compositional aspects of a first volatile composition of the first volatile composition dispenser;
   d. determining compositional aspects of a second volatile composition of the second volatile composition dispenser; and
   e. adjusting, via the controller, at least one dispensing parameter of at least one of the first volatile composition dispenser and the second volatile composition dispenser based on at least one of the spatial relationship, the compositional aspects of the first volatile composition, and the compositional aspects of the second volatile composition.
B. The method of according to Paragraph A, wherein adjusting the at least one dispensing parameter comprises adjusting temperature settings of the first volatile composition dispenser or the second volatile composition dispenser.
C. The method according to any of Paragraphs A or B, wherein the spatial relationship comprises a distance between the first volatile composition dispenser and the second volatile composition dispenser, wherein the distance is between about 3 meters and about 50 meters.
D. The method according to any of Paragraphs A to C further comprising determining, via the ultra-wideband radar data, a position of at least one object between the first volatile composition dispenser and the second volatile composition dispenser, and adjusting the at least one dispensing parameter of the first volatile composition dispenser based on the position of the at least one object.
E. The method according to any of Paragraphs A to D further comprising adjusting the at least one dispensing parameter based on a compatibility score of the first volatile composition and the second volatile composition.
F. The method according to Paragraphs A to E further comprising adjusting at least one lighting characteristic of at least one visual indicator of the first volatile composition dispenser based on at least one of the spatial relationship, the compositional aspects of the first volatile composition, and the compositional aspects of the second volatile composition.
G. The method according to Paragraph F, wherein the at least one lighting characteristic includes at least one of color, intensity, and direction.

While devices, systems, compositions, or methods are described herein in terms of "comprising" various components or steps, the devices, systems, compositions, or methods can also "consist essentially of" or "consist of" the various components or steps, unless stated otherwise.

As used herein, the word "or" when used as a connector of two or more elements is meant to include the elements individually and in combination. For example, "X or Y" means X or Y or both.

As used herein, the articles "a" and "an" are understood to mean one or more of the material that is claimed or described, for example, "a volatile composition" or "a connector", unless stated otherwise.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as "40 mm" is intended to mean "about 40 mm."

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests, or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A volatile composition dispenser comprising:
a transceiver; and
a controller, wherein the controller is configured for:
determining a spatial relationship of the volatile composition dispenser relative to at least one of a surrounding environment and a second volatile composition dispenser; and
modifying at least one dispensing parameter based on the spatial relationship;
wherein the volatile composition dispenser is configured to dispense at least one volatile composition based on the at least one dispensing parameter.

2. The volatile composition dispenser of claim 1, wherein the transceiver comprises an ultra-wideband transceiver configured to transmit and receive ultra-wideband radar data, wherein the spatial relationship is determined based on the ultra-wideband radar data.

3. The volatile composition dispenser of claim 1, wherein the volatile composition dispenser comprises a cartridge, wherein the cartridge comprises a unique identifier corresponding to a respective composition of the at least one volatile composition, wherein the controller is configured to identify fragrance notes in the respective composition based on the unique identifier.

4. The volatile composition dispenser of claim 3, wherein the controller is configured to adjust the at least one dispensing parameter based on identified fragrance notes in the at least one volatile composition.

5. The volatile composition dispenser of claim 1, wherein the controller is further configured for modifying the at least one dispensing parameter based on a volatile composition housed and dispensed by the second volatile composition dispenser.

6. The volatile composition dispenser of claim 1, wherein the surrounding environment comprises distance data and directional data related to at least one object in a proximity of the volatile composition dispenser.

7. The volatile composition dispenser of claim 1, wherein the volatile composition dispenser further comprises at least one visual indicator, wherein the controller is configured to adjust at least one lighting characteristic of the at least one visual indicator based on at least one of the spatial relationship and compositional aspects of the at least one volatile composition.

8. A volatile composition dispensing system comprising:
a first volatile composition dispenser comprising a first volatile composition, a first ultra-wideband transceiver, and a first controller;
a second volatile composition dispenser comprising a second volatile composition, a second ultra-wideband transceiver, and a second controller;
wherein the first controller is configured for modifying at least one dispensing parameter of the first volatile composition dispenser based on at least one of:
a spatial relationship between the first volatile composition dispenser relative to the second volatile composition dispenser;
compositional aspects of the second volatile composition;
a compatibility score of the first volatile composition and the second volatile composition; and
a unique identifier corresponding to the first volatile composition that is wirelessly communicated to the first controller.

9. The volatile composition dispensing system of claim 8, wherein the second controller is configured for modifying at least one dispensing parameter of the second volatile composition dispenser based on at least one of the spatial relationship between the first volatile composition dispenser and the second volatile composition dispenser, compositional aspects of the first volatile composition, and the compatibility score of the first volatile composition and the second volatile composition.

10. The volatile composition dispensing system of claim 8, wherein the first controller is configured to adjust the at least one dispensing parameter based on at least two of the spatial relationship between the first volatile composition dispenser and the second volatile composition dispenser, compositional aspects of the second volatile composition, the compatibility score of the first volatile composition and the second volatile composition, and the unique identifier corresponding to the first volatile composition.

11. The volatile composition dispensing system of claim 8, wherein the first controller is configured to adjust the at least one dispensing parameter based on at least three of the spatial relationship between the first volatile composition dispenser and the second volatile composition dispenser, compositional aspects of the second volatile composition, the compatibility score of the first volatile composition and the second volatile composition, and the unique identifier corresponding to the first volatile composition.

12. The volatile composition dispensing system of claim 8, wherein the at least one dispensing parameter comprises at least one of a temperature setting, an activation of a fragrance note, and a deactivation of a fragrance note.

13. The volatile composition dispensing system of claim 8, wherein the first controller operates the first volatile composition dispenser at a first temperature range, wherein the second controller operates the second volatile composition dispenser at a second temperature range different from the first temperature range, wherein the first temperature range and the second temperature range are based on at least one of the spatial relationship and the compatibility score of the first volatile composition and the second volatile composition.

14. The volatile composition dispensing system of claim 8, wherein the spatial relationship comprises distance data and directional data related to an object positioned between the first volatile composition dispenser and the second volatile composition dispenser.

15. The volatile composition dispensing system of claim 8, wherein the first volatile composition dispenser comprises a cartridge, wherein the cartridge comprises the unique identifier corresponding to the first volatile composition, wherein the first controller is configured to identify fragrance notes of the first volatile composition based on the unique identifier.

16. The volatile composition dispensing system of claim 8, wherein
the first volatile composition dispenser further comprises at least one visual indicator, wherein the first controller is configured to adjust at least one lighting characteristic of the at least one visual indicator based on at least one of the spatial relationship, compositional aspects of the second volatile composition, the compatibility score of the first volatile composition and the second volatile composition, and the unique identifier corresponding to the first volatile composition.

17. A method of volatile composition dispensing using the volatile composition dispensing system of claim 8, the method comprising:
capturing, via the first ultra-wideband transceiver of the first volatile composition dispenser, ultra-wideband radar data;
determining, via the first controller operably connected to the first ultra-wideband transceiver, a spatial relationship between the first volatile composition dispenser and the second volatile composition dispenser, wherein the spatial relationship is determined based on the ultra-wideband radar data;
determining compositional aspects of the first volatile composition of the first volatile composition dispenser;
determining compositional aspects of the second volatile composition of the second volatile composition dispenser; and
adjusting, via the first controller or the second controller, at least one dispensing parameter of at least one of the first volatile composition dispenser and the second volatile composition dispenser based on at least one of the spatial relationship, the compositional aspects of the first volatile composition, and the compositional aspects of the second volatile composition.

18. The method of claim 17, wherein adjusting the at least one dispensing parameter comprises adjusting temperature settings of the first volatile composition dispenser or the second volatile composition dispenser.

19. The method of claim 17, further comprising determining, via the ultra-wideband radar data, a position of at least one object between the first volatile composition dispenser and the second volatile composition dispenser, and adjusting the at least one dispensing parameter of the first volatile composition dispenser based on the position of the at least one object.

20. The method of claim 17, further comprising adjusting the at least one dispensing parameter based on a compatibility score of the first volatile composition and the second volatile composition.
